(19) United States
(12) Patent Application Publication
(10) Pub. No.: US 2010/0221144 A1
(43) Pub. Date: Sep. 2, 2010

(54) AIR FRESHENER KIT

(75) Inventors: Gregory W. Bedson, Scottsdale, AZ (US); Deborah J. Cowan, Scottsdale, AZ (US); Geoffrey Faires, Cave Creek, AZ (US); Jeff Gaunt, Phoenix, AZ (US); Eric J. Horsman, Phoenix, AZ (US); Nancy Miiller, Scottsdale, AZ (US); Elizabeth Polaski, Scottsdale, AZ (US); Keith Queen, Phoenix, AZ (US); Judith A. Wallis, Chandler, AZ (US); Cheryl Watkins, Scottsdale, AZ (US)

Correspondence Address: THE DIAL CORPORATION, 19001 N. Scottsdale Road, SCOTTSDALE, AZ, 85255, US

(73) Assignee: The Dial Corporation, Scottsdale, AZ (US)

(21) Appl. No.: 12/503,154

(22) Filed: Jul. 15, 2009

Related U.S. Application Data
(60) Provisional application No. 61/156,581, filed on Mar. 2, 2009.

(51) Int. Cl. A61L 9/12; A61L 9/04; B65B 7/02

(52) U.S. Cl. 422/4; 422/120; 53/480

(57) ABSTRACT

The present invention comprises a kit for assembling a passive air freshener comprising fragranced sail crystals. In the preferred form, the kit minimally comprises: 1) a package containing a plurality of fragranced salt crystals; 2) a container with an opening for placing said crystals therein, said container defining an enclosed space having a height/width ratio of from about 0.5 to about 3; and, 3) a carton for containing both the package of crystals and the container, where the carton preferably has a viewing window that displays at least the decorative aspects of the container design to potential purchasers. The kit may also include instructional literature and optional lighting source.

### PRIORITY APPLICATION

The present application claims priority to U.S. Provisional Application 61/156,581 filed Mar. 2, 2009 and entitled “FRAGRANCED SALT CRYSTAL AIR FRESHENER AND KIT FOR ASSEMBLING SAME”, which is incorporated herein.

### FIELD OF INVENTION

The present invention relates generally to air fresheners and in particular to a kit for assembling a passive salt crystal air freshener comprising a carton that includes a package of fragrance coated salt crystals along with an appropriately sized container in which to place the crystals for use.

### BACKGROUND

Air fresheners have been in the marketplace and in the patent literature for many decades. Consumer air fresheners run the gamut from static blocks of fragranced gel to programmable and playable electronic devices with heaters and/or fans, and correspondingly are sold across a wide price range. Other products are simple aerosols, such as the Renuzit® Subtle Effects™ aerosol sprays. “Passive” refers to air fresheners that do not require any energy to operate. That is, a passive air freshener can scent a room simply sitting at ambient temperature in a home. These passive air freshener products release volatile fragrance components from a solid matrix, pad, or other carrier over time, with the evaporation rate (and product length of life) usually determined by the volatility of the fragrance mixture, the surface area from which the fragrance mixture can evaporate, and the extent to which airflow can interact with the product. Some passive air fresheners rely on emanating pads or wicks having very large surface areas to evaporate a volatile liquid fragrance mixture into the room. Other typical passive air fresheners are static blocks of fragranced gel, such as the Renuzit® Adjustable Cone air freshener that has been in the market for many years.

Other examples of passive air fresheners include fragranced salt granules (e.g. loose or in vapor-permeable sachets), fragranced mineral rocks or mined crystalline materials, and molded, tableted or pelletized salts admixed with fragrances. These variations are described in a number of exemplary references including: U.S. Pat. No. 3,371,984 (Kelly, et al.); U.S. Pat. Nos. 5,041,421 and 5,246,919 (King); U.S. Pat. No. 6,270,720 (Mandish); U.S. Pat. No. 6,426,325 (Dente, et al.); and, U.S. Patent Application Publication 2006/0293215 (Sizemore). The King patents ('421 and '919) describe a variety of fragranced salt products, including fragranced salt pellets and molded/tabletized material formed from compression of scented salt granules in a tablet press. Sizemore (in U.S. Patent Application Publication 2006/0293215) states that salt crystals are not porous to fragrance oils (PARA [0003]) and consequently discloses an idea to use electromagnetic radiation to improve the coating of salt crystals. Examples of salt crystal air fresheners may also be found on the Internet under the general descriptions of “crystal potpourri” or “crystal rock potpourri”. These products generally appear to be mined rocks or sea salt rocks, with some advertisers suggesting that the rocks be placed in a simmering pot or other heated fragrance oil potpourri bowl. With little guidance, the purchaser of bulk scented salt rocks may use (or misuse) the rocks any way they wish.

Air freshener “kits” that can guide consumers to build their own air freshening system number few in the literature. U.K. Patent Application Publication GB 2199246 to Lam discloses an air freshener kit comprising a plurality of scented wax chips and a heating vessel in which to melt the chips. U.S. Patent Application Publication 2008/0286143 to Grodsky discloses a kit comprising a container, wick, fragrance and snuffer cap useful for assembling a passive diffuser-type air freshener. Finally, PCT Application Publication WO 01/68155 to Requejo discloses a fragranced hydrogel air freshener kit that includes a packet of acrylic polymer, fragrance and colorant along with a vessel in which the consumer mixes these ingredients together with water to form a gel air freshener.

What is clearing lacking in the literature is an air freshener kit for assembling an air freshener that comprises scented salt crystals. Without a kit to guide a consumer, it may not be clear how to use fragranced salt crystals that have aesthetic beauty worthy of display and consumer-acceptable fragrance strength to enjoy.

### SUMMARY OF THE INVENTION

The present invention comprises a kit for assembling a passive salt crystal air freshener. In the preferred embodiment, the kit minimally comprises: 1) a plurality of fragranced salt crystals within a package; 2) a container in which to place the crystals for use; and, 3) a carton for containing both the package of crystals and the container, wherein the carton preferably includes a viewing window that provides for display of at least part the container (and optionally the crystals package) to potential purchasers.

The fragranced salt crystals included in the kit of the present invention comprise large, natural, evaporative-sou reed salt crystals sieved to range in size from about ⅜ inch to about 1 inch, tumble-coated with a combination of fragrance oil, fumed silica, and an emulsifier. The size range of the salt crystals has been found to be critical in maximizing the fragrance perception from the crystals when they are placed in an open jar. Preferably a plurality of the fragranced salt crystals is sealed within a flexible package, (e.g. a flexible plastic or aluminum vapor-impermeable bag or pouch, optionally re-closable with a tie or a zipper).

The second component of the kit comprises any suitable open top container such as a decorative jar in which the consumer may place the fragranced salt crystals. It has been discovered that the scented salt crystals give maximum and consistent fragrance delivery when placed in an open top container having a height/width ratio of from about 0.5 to about 3. Optionally the open top container may include a lid or other feature usable to partially or fully close off the opening of the container.

Lastly, both the package of fragranced salt crystals and the container are housed inside a carton that allows for the shipping, storage, display and promotion of the air freshener product. The carton preferably features a view window such that at least part of the container is visible through the window. Instructions for assembling a passive salt crystal air freshener using the present kit may be printed directly on the inside or outside of the carton or included as separate literature inside the carton.

### BRIEF DESCRIPTION OF THE DRAWINGS

FIG. 1 illustrates an air freshener kit in accordance with the present invention.

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the relative sizes and shapes of the components of the kit described herein without departing from the scope of the invention as set forth in the appended claims. For example, changes in the structure of the carton, such as its shape (cubic, rectangular cuboid, gabled, etc.) or its salient features, (with viewing window, with handles, etc.), or its materials of construction (cardboard, wood, metal, plastic), are within the scope of the invention. Also, the shape of the crystals, as possibly achieved by switching between various methods for evaporation of the brine (solar, ambient, heat, or combinations thereof), or the source for the brine (sea water, Great Salt Lake water, brine wells, or manufactured saturated salt water solutions) are within the scope of the invention. Although the present invention is described in utilitarian terms as a kit for assembling a passive air freshener comprising fragranced “natural salt” crystals, no restriction as to the chemical makeup of the crystals, or the source of the starting brine, is herein implied. Most preferred is to use larger natural sea salt crystals as obtained through evaporation of natural sea or Great Salt Lake water, (via solar, wind, ambient air or heated methods of evaporation). Lastly, changes to the shape, size, color, or materials of construction for the container are within the spirit of the invention. There exists near infinite variations of the container included within the kit of the present invention. The container may be made from a variety of materials (glass, plastic, metal, rattan, weaved twigs, ceramic or clay pottery, etc.), it may be clear, opaque, colored, white, natural, etc., it may be round, tall, short, rectangular, etc., and it may include useful features such as child-resistant screen/grating to cover the open top, a saucer/coaster with or without a lighting feature for underneath, one or more handles, etc., as long as the container can hold the fragranced salt crystals and allow the scent of the crystals to permeate out, any design for the container is within the scope of the present invention.

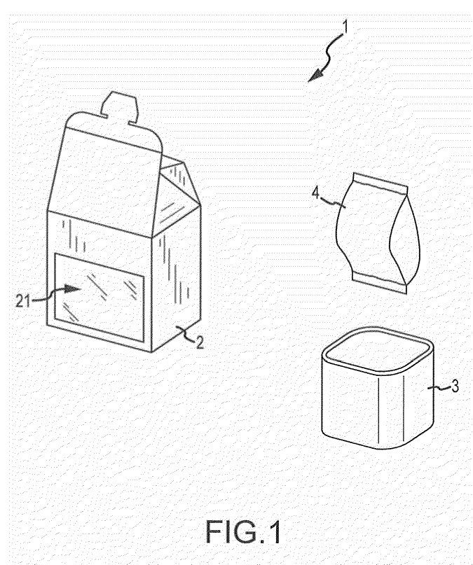

That said, the present invention relates to a kit for assembling a passive salt crystal air freshener comprising a package containing a plurality of evaporation-sourced salt crystals coated with a fragrance pre-blend, a container in which said crystals may be placed, and a carton for holding both the package of salt crystals and the container. More specifically the present invention is an air freshener kit comprising: (a) a package containing a plurality of fragranced salt crystals further comprising (i) evaporative-sourced salt crystals with size distribution falling between about ⅜ inch and about 1 inch U.S. sieve sizes, (ii) fragrance oil, (iii) fumed silica, and (iv) a nonionic emulsifier; a container wherein the crystals may be placed; and, a carton for containing both the package of said crystals and the container. Referring now to FIG. 1, the kit 1 of the present invention comprises: a package 4 containing a plurality of fragranced salt crystals; an open container 3; and, a carton 2 for containing both said package and said container. The carton 2 preferably includes a viewing window 21 that provides a view of at least part of the container 3 and/or package 4. When the package 4 is transparent, it may be preferred to have some of the package visible through window 21 such that the consumer can see the crystals as well as the container 3.

The method of manufacture for the present invention comprises the steps of pre-mixing of the fumed silica with the fragrance, next adding the emulsifier (and optional dyes) and then using this fragrance premix to coat the salt crystals. A plurality of the fragranced and optionally dyed crystals, (e.g. 10 g to 1 kg), is then packaged in a suitable package such as a plastic pouch. A container with an opening, such as ajar, is selected for inclusion in the kit, and both the container and bag of fragranced salt crystals are placed into the carton. Optional instructional literature may be included in the carton as well.

The method of use for the present invention is to remove the contents of the carton, open the package of fragranced salt crystals, place some or all of the fragranced salt crystals into the open container, attach the optional cover/grill onto the container opening if supplied, and place the container with the salt crystals in the area to be scented (e.g. a room in a home). The container with the crystals arranged therein provides aesthetic beauty and optimal fragrance delivery. The optional grill on the container opening provides some child resistance. An optional solid cover provides a means to close off the container and stop the scenting of the space where the container has been placed. For example the container may be an urn and may be supplied with a lid that can be set on top to close off exposure of the salt crystals from the air.

Evaporative-Source Salt Crystals

Salt crystals suitable for use in the present air freshener comprise crystals that are obtained through evaporation of a salt/brine solution comprised primarily of sodium chloride (NaCl), potassium chloride (KG) or mixed salt brine. Herein “brine” is taken to be a chloride salt solution, (i.e. M⁺Cl⁻, where M⁺ is Na⁺, K⁺, Mg²⁺, etc.). Preferably the crystals are simply natural sea or lake salt crystals that are obtained by evaporation of Atlantic, Pacific or Caribbean ocean waters, or Great Salt Lake water, through various well known methods (e.g. solar, ambient air, wind, heat, or combinations of evaporative methods, some depending on weather). Sea salt crystals are available from many suppliers such as Cargill, Morton, ESSA (Exportadora de Sal) or Diamond, and most preferred is to use “solar salt” obtainable from Cargill (Caribbean) or ESSA (Pacific). These sea salt crystals are grown in open ponds of sea water exposed to the sun. Also of use in the present invention are salt crystals grown from evaporation of brine well water. Additionally, ordinary salt (e.g. powdered or granulated NaCl, KCl, or mixed salts) could be dissolved in water to form a saturated solution and that solution could then be used to grow crystals useful for the present invention. Regardless of the source of the brine to evaporate, the salt crystals for use in the present invention preferably range in size from about ⅜ inch to about 1 inch long, as obtained by subsequent sieving of salt crystals with ⅜ inch and 1 inch U.S. Standard sieves. It is advantageous to have crystals that give a high yield of this size range when sieved, (i.e. a minimum of both “fines” and huge “rock-like” crystals). The crystals to be scented herein are not mined minerals (i.e. not rock salt or halite). Nonetheless, crystal shapes from evaporation of brine vary considerably, and this natural distribution of shapes and sizes is desirable for this air freshener. In fact, solar salt crystals grown from ocean waters have variability simply because of the daily weather variation (hot sunny days, cold days, windy days, etc.) and the influence of other minerals that affect crystal growth, number of layers and clarity.

As mentioned, a distribution of crystal size is preferably used herein, and that distribution is obtainable by collecting the crystals that remain trapped between stacked 1 inch and ⅜ inch U.S. Standard sieves. The remains on top of the 1 inch sieve (huge “rock-like” crystals), and what falls all the way through both sieves (the “fines”), is discarded. Of course, unusually long and narrow shaped crystals may manage to slip through the 1 inch sieve, and those narrow crystals may have lengths of up to 2 to 4 inches. These rare crystals arc still useful for this present invention because they tend to break up into smaller crystals during the fragrance coating process (i.e. during tumbling). As mentioned, the crystals trapped in between these two U.S. Standard sieves are ideal for the present invention. This “cut” ranges in size, shape, color, and clarity, giving uniqueness to the air freshener product. Since it is difficult to describe the shapes of the natural crystals, the preferred size range for use herein is defined as a sieve range.

Using crystals that measure less than about ⅜ inch (i.e. crystals that pass through both sieves) will lead to fragranced crystals that pack too uniformly and tightly when placed in the open container provided in the kit (or any other container supplied by the consumer). When the fragranced crystals pack tightly, there is insufficient air flow between the crystals and an unacceptable fragrance delivery results. The preferred size range of from about ⅜ inch to about 1 inch, with some distribution in both size and shape, ensure that the fragranced decorative crystals will produce a consumer acceptable fragrance intensity when placed in an open jar. Thus, for the present invention, it is most preferred to use natural sea salt (e.g. “solar salt”) having a size distribution falling between about ⅜ inch and about 1 inch sieve sizes. Or in other words, it is most preferred to use the sieved “cut” of natural solar sea salt crystals that remain between stacked ⅜ inch and 1 inch U.S. Standard sieves, (i.e. crystals that has pass through the 1 inch screen but are stopped by the ⅜ inch screen).

Fumed Silica

Commercially available fumed silica (or silica fume) may be formed from the conversion of silicon tetrachloride with hydrogen and oxygen in the gas phase (e.g. flame process). Commercial fumed silica is sub-micron (e.g. nanometer), very pure silicon dioxide (SiO₂). It may be purchased under the trade names Aerosil® by Evonik-Degussa and Cab-O-Sil® from Cabot Corp. Particularly useful for the fragrance pre-mix described herein is the “untreated” or hydrophilic fumed silica such as Cab-O-Sil® M-5 or Aerosil® 200 (both general purpose untreated fumed silica having moderate surface area of about 200 m²/gram). Fumed silica traditionally finds use in liquids for rheology control, suspension, moisture resistance, viscosity stability and emulsification. For pre-blends comprising fragrance oil, nonionic emulsifier and fumed silica, the silica may supply most if not all of these functions. The fragrance pre-blend preferably comprises from about 0.1% to about 10% by weight fumed silica. Most preferred is to prepare a pre-blend comprising from about 0.1% to about 5% by weight fumed silica. The air freshener of the present invention (the finished salt crystals coated with a fragrance pre-blend) preferably comprises from about 0.001% to about 2% by weight fumed silica. Most preferred is to have the fumed silica at a level of from about 0.05% to 0.15% by weight of the finished scented salt crystals.

Emulsifier

The emulsifier for use in the present invention may comprise at least one nonionic material including polysorbates, polyalkylene glycols, fatty alcohols, and various nonionic surfactants and stabilizers, and mixtures thereof. Preferred nonionic emulsifiers for use herein include the polysorbates, such as the Span®, Brij® and Tween® products from Croda (formerly Uniqema). These materials are sorbitan derivatives comprising a polyethoxylate chain and a fatty chain.

Preferred nonionic surfactants include ethoxylated, propoxylated, or mixed ethoxylated/propoxylated; alkylphenol ethers, linear aliphatic or fatty alcohols C4-C16, mono- and di-esters of aliphatic C4-C16 carboxylic acids, branched aliphatic alcohols with a main aliphatic carbon chains of C4-C16, hydrogenated castor oils (such as the Cremophor® materials from BASF) and glycols; and ethoxylated hydrogenated castor oil monopyroglutamic monoisostearic diesters, ethoxylated glycerol monopyroglutamic moniostearic diesters, and other pyrrolidon carboxylic acid derivatives. A preferred ethoxylated aliphatic alcohol for use in the present invention is Tomadol® 25-12, from Tomah, which is essentially C12-C15 alcohol with an average 12 moles ethylene oxide. Also preferred is Eumulgin® HPS from Cognis, which is a mixture of ethoxylated alcohols, EO/PO glycol ethers, and ethoxylated hydrogenated castor oil, and Genapol® products from Clariant. Other preferred nonionic surfactants include the amine oxide surfactants. The preferred amine oxide surfactant for use in the present invention is typically a trialkyl amine oxide, most preferably an alkyldimethylamine oxide. Examples of such materials that find use in the composition are Ammonyx® LO from Stepan, Barlox® 12 from Lonza Corporation, and Surfox® LO Special from Surfactants, Inc. These compounds are essentially aqueous or water/alcohol solutions of lauryl- or myristyl-dimethylamine oxide or blends/chain length distributions thereof. Of particular use, and one which has worked for several fragrance types, is a mixture of an ethoxylated alcohol and an amine oxide, and most preferred is to use combinations of Tomadol® 25-12, Surfox® LO Special and Cremophor RH-40, each from about 0.25% to about 7% and in total in the present invention from about 2% up to about 10%.

Fragrance Oil

The fragrance in the composition of the present invention may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abics oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanuni res., onion oil, opoponax res., orange oil, orange flower oil. origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolti balsam, tonka beans abs., tuberose abs., turpentine oil, vanilla beans abs., vetiveroil, violet leaf abs., ylang ylang oil and like vegetable oils, etc. Synthetic fragrance materials include but are not limited to pinene, limonenc and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, .gamma.-nonyl lactone, coumarin, cincole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce a number of overall fragrance type perceptions including but not limited to, fruity, musk, floral, herbaceous (including mint), and woody, or perceptions that are in-between (fruity-floral for example). Typically these fragrance mixtures are compounded by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof, and using solvent up to as much as 90% of the volatizable fragrance composition. The preferred fragrance oil for use in the salt crystal air freshener of the present invention may be comprised of a mixture of many fragrance actives and volatile solvents, sometimes along with smaller amounts of emulsifiers, stabilizers, wetting agents and preservatives. More often than not, the compositions of the fragrance oil purchased from the various fragrance supply houses remain proprietary and thus can only be described in general terms. The fragrance oil is preferably incorporated at a level of from about 80% to about 99.8% by weight in the fragrance premix, and from about 0.8% to about 16% by weight of the total weight of the finished fragranced salt crystals.

Optional Adjuvant

The air freshener crystals of the present invention may also include dyes, pigments or other suitable colorants to provide aesthetic appeal to the fragranced crystals. Such dyes may include FD&C and/or D&C Yellows, Reds, Blues, Greens and Violets, or really any other dye or pigment, and such materials are commonly purchased in powder or liquid form. Dyes and/or pigments are incorporated at levels sufficient to provide light color to deep color to the finished crystals. When dyes or other pigments are desired for colored air freshener salt crystals, they are included in the fragrance premix at from about 0.0001% to about 1% by weight, depending on the concentration of the colorants (e.g. if a liquid/diluted dye or a neat powder).

The fragranced salt crystal air freshener of the present invention may also include one or more preservatives to help prevent dye fading and mold and other microbial growth on the crystals. The preferred microbial preservatives include Neolone® and Kathon® products from Lonza and Rohm & Haas. These materials are incorporated at the manufacturers' recommended levels in the fragrance premix to discourage bacterial and mold growth on the finished crystals. An ultraviolet inhibitor and/or an antioxidant may also be added to the fragrance premix to reduce dye fade on the finished crystals.

The present invention may also include one or more solvents to aid in control of viscosity of the fumed silica/emulsifier/fragrance oil premix. Some of these solvent materials overlap with what was defined as nonionic emulsifiers above, and include, but are not limited to, ethanol, methanol, isopropanol, n-propanol, n-butanol, MP-Diol (methylpropanediol), ethylene glycol, propylene glycol, and various glycol ethers (e.g. ethylene or diethylene glycol monoalkyl ethers, and propylene or dipropylene glycol monalkyl ethers, and the like), and mixtures thereof. These solvents may be used in any combination at a level of from about 0.1% to about 5% by weight in the premix to adjust viscosity such that the fragrance premix coats the salt crystals evenly. As mentioned, any of these solvents may be present in the proprietary fragrance oil as obtained from any number of fragrance suppliers, and it is possible to have the fragrance oil supplier add the viscosity reducing solvent to the purchased fragrance oil rather than for the end formulator to put it into the fragrance premix.

The Processing Methods

The preferred method for producing the fragranced crystals of the present invention involves a first step of producing a pre-blend. The pre-blend preferably comprises from about 80% to about 99.8% by weight fragrance oil; from about 0.1% to about 10% by weight fumed silica; and, from about 0.1% to about 10% by weight emulsifier, along with optional dyes and other adjuvant. Most preferred is to prepare a pre-blend comprising from about 90% to about 99% by weight fragrance oil; from about 0.1% to about 5% by weight fumed silica; and, from about 0.1% to about 5% by weight emulsifier, along with optional dyes and other adjuvant. When dyes or other pigments are desired, they are included in the pre-blend at from about 0.0001% to about 1% by weight, depending on the concentration of the colorants (e.g. if a liquid/diluted dye or a neat powder). The fragrance oil, fumed silica and the optional dyes are first mixed together to form a wet silica paste. The emulsifier is then added and mixed thoroughly in to produce a thickened liquid pre-blend that is used to coat the salt crystals. Thicker pie-blends may be made by increasing the weight percent of fumed silica in the pre-blend.

The second step for producing the fragranced crystals of the present invention is to use the above described pre-blend to coat the salt crystals. Preferably a mixture is prepared in the blender comprising from about 80% to about 99% by weight of salt crystals and from about 1% to about 20% by weight of the above described pre-blend. Most preferred is to blend 95% to about 99% salt crystals by weight with from about 1% to about 5% by weight fragrance pre-mix. As mentioned, the preferred salt crystals are evaporative-sourced, and most preferably comprise natural sea salt crystals having a largest dimension of from about ⅜ inch to about 1 inch. It is most preferable to have a distribution of crystal sizes to reduce uniform packing of the air freshener crystals in use (e.g. in a jar) and to increase visual aesthetics. A “V-blender”, rolling drum blender, “double-cone” blender or other suitable tumble-coating machinery is charged with the appropriate amount of salt crystals for the batch. The pre-blend is then added to the salt crystals and the mixer is turned on. The crystals are tumbled with the fragrance pre-mix until there is even coating of the crystals hut not excessive breakage. It has been found that the “V-blender” and the “double cone” blender provide the best coating of the salt crystals without concomitant breakage of crystals.

Given the preferred weight percentages in the pre-blend and the preferred weight percentages of the pre-blend/crystals blending, it follows that the preferred weight percentages of the final air freshener crystals comprise: from about 80% to about 99% salt crystals; from about 0.8% to about 16% fragrance oil; from about 0.001% to about 2% fumed silica; and, from about 0.001% to about 2% emulsifier. It is most preferred that the final fragranced salt crystals of the present invention comprise: from about 95% to about 99% by weight natural sea salt crystals; from about 1% to about 5% by weight fragrance oil; from about 0.05% to about 0.15% fumed silica; and, from about 0.005% to about 0.015% emulsifier.

Compositions of the fragranced salt crystals included in the kit of the present invention are shown in TABLE 1.

TABLE 1
Fragrance Premix and Finished Fragranced Salt Crystals

| | Weight % | |
|---|---|---|
| Component | Fragrance Premix | Fragranced crystals³ |
| Natural sea salt crystals¹ | — | 97.000 |
| Fragrance oil | 96.5 | 2.895 |
| Fumed Silica | 3.0 | 0.090 |
| Emulsifier² | 0.3 | 0.009 |
| Optional Dyes | 0.2 | 0.006 |

Notes:
¹Atlanta, Caribbean or Pacific Ocean natural solar salt crystals with distribution from about ⅜ inch to about 1 inch obtained by U.S. Standard sieves;
²Tween ® 80, Tween ® 60 or Tween ® 20, or mixtures thereof;
³Final composition based on blending 3 wt. % fragrance premix with 97 wt. % solar salt crystals.

Packaging for the Plurality of Fragranced Salt Crystals

As mentioned, the air freshener kit of the present invention preferably includes packaging suitable to stably contain a plurality of the fragranced salt crystals described, (FIG. 1, element 4). That is, the above described fragranced salt crystals are preferably contained in a primary package and that package is included in the secondary carton 2. The fragrance oil on the crystals has tendency to wick into and stain absorbent materials such as the display carton 2. Also, loose fragranced crystals, left unpackaged, will lose fragrance strength. Therefore it is preferable to place a plurality of the fragranced crystals into a vapor-impermeable package such as a plastic and/or metallic laminated bag or pouch that will help retain the color, integrity and fragrance of the fragranced crystals. Another packaging option is to pre-fill the container 3 with the fragranced salt crystals and seal the filled container with a cover or a plastic shrink wrap for merchandizing. In practice, supplying the container 3 pre-filled with the fragranced salt crystals turned out to be inferior. The fragrance oil and dyes on the crystals wet and stained the inside of the container and was found to be especially unacceptable when the container 3 was made of clear glass. Additionally, the staining and wetting of the inside walls of the container 3 worsened when the pre-filled container was placed in shipping tests. However, it's possible that for other types of containers, (i.e., not clear glass), pre-filling the container and shrink wrapping it may still be a useful option. For example, a small wicker basket pre-filled with fragranced crystals and shrink-wrapped in clear plastic may make a desirable merchandisable unit without need for additional packaging.

Film-type packaging for the fragranced salt crystals may be comprised of a number of laminated materials common in the marketplace. For example, bags or pouches including gusseted pouches may be produced from the materials and processes disclosed in U.S. Pat. No. 5,846,620 (Compton) and U.S. Pat. No. 4,598,826 (Shinbach), and U.S. Patent Application Publication 2005/0255200 (Takahagi, et al.). Simple sealable/resealable plastic “sandwich bags”, metal film bags (e.g. aluminum), and plastic/metal laminated bags and pouches are all suitable packaging in which to stably contain the plurality of fragranced crystals. The packaging may be heat sealed and there is the option of including a reseating strip (e.g. a thermoplastic zipper structure). The amount of crystals provided in the packaging 4 is greatly variable. For example, there may be just enough fragranced salt crystals provided in the packaging 4 to fill the container 3 about once. Alternatively, a surplus of crystals may be provided such that the container 3 can be filled twice or more times. When supplying a surplus of crystals usable to refill the container 3 more than once, the use of a resealable feature on bag 4 becomes preferred. Preferably the amount by weight of fragranced salt crystals within package 4 is from about 10 grams to about 5 kilograms. Certainly, the bags of crystals can be marketed and sold separately as refill pouches of fragranced salt crystals, in which case the consumer keeps the container 3 from the starter kit, and thereafter purchases only refill bags of crystals 4 in order to keep refilling the same container. The container can be washed out between refills to maintain pleasing aesthetics.

The Container

The container included in the kit of the present invention may be molded or blown glass, plastic, metal, unglazed pottery or ceramics (e.g. terracotta, greenware, bisque, etc.), glazed ceramic, or weaved from twigs, yarn, reed, grasses, rattan, wicker, and the like. The terms “container” and “container having an opening” used herein means an open vessel, like ajar, vase, glass, bowl, pot, basket, bucket/pail, mug, box, urn, or dish, having a bottom, sidewalls and an open top, where the solid surfaces contiguously mate together to define an enclosure having an interior space or volume. The container may be short in height and shaped more like a “dish” or may be tall and shaped more like ajar, urn, or vase. It may have distinct flat sides with sharp angles or may be round or cylindrical. It may have features like a handle on top to resemble a basket or pail, or on the side as a coffee mug. Clear glass or plastic, blown or molded, arc the preferred materials of construction for the container, although more artistically elaborate containers include such things as small weaved baskets, coffee mugs, flower pots, or small tin boxes or pails, all having utility in the present invention. Most preferred is that the open container included in the kit have a height/width ratio of from about 0.5 (i.e. 1:2) to about 3 (i.e. 3:1) or so. That is, very flat dishes or plates are not desirable, nor are really tall, narrow vases. We have found that fragrance delivery from the fragranced crystals made as described above is best from a container having a height/width ratio of from about 0.5 to about 3, and most preferred from about 1:1 to about 2:1. A preferred container for use in the present invention is shown in U.S. Design Pat. D484,365 (Wyche). Another preferred container is shown by U.S. design D502,101 (Kerman), which has a lid that can be used to close off the container of crystals when fragrance emanation is no longer desired. Of course, stemware or other containers may be used that are raised up on a pedestal or stand of sorts that is molded into the design, provided that the enclosed area where the crystals arc placed has the preferred height/width ratio of from about 0.5 to about 3 or so. Thus, a large martini glass or margarita glass may be used as the container of the present kit, where the stem or pedestal is ignored in the calculation of the preferred dimensions. A theme can be set up whereby the fragrance of the crystals is linked to the visual elements of the container provided. For example, floral scented salt crystals paired with a flower pot as the container; coffee, vanilla or chocolate scented salt crystals paired with a coffee mug shaped container; spice scented salt crystals paired with a wicker basket as the container; ocean/airy scented salt crystals paired with a small aluminum pail; or, fruit scented salt crystals paired with Ball®/Mason® type canning jars, etc. The preferred volume of the container is from about 100 mL to about 5 Liters. Size may be chosen on the basis of decorative theme, and/or weight of crystals initially supplied in the kit, and/or weight of crystals supplied in separately sold refill packages. Of course, an elaborate and expensive container may be provided to support a marketing theme of “collectables”, or simply to encourage future purchase of refill packages of crystals. On the other extreme, the container may be very inexpensive glass or plastic, even to the point of being disposable with no thought of a refillable execution.

As mentioned, some type of lid to cover the opening of the container is an option. In the extreme, the lid may be a solid cover that sits or snaps over or screws onto the container opening to seal it completely. Such a lid may be hinged to the container or supplied as a separate item. The lid may be the same material as the container or different. For example, a ceramic urn may be supplied with a matching glazed ceramic lid. A glass or ceramic container resembling a jar may be supplied with a flexible plastic lid (e.g. made from thin PE or PP) that snaps over the opening of the jar, or a metal screw-cap lid that threads onto the opening of the jar. A metal container, such as one made from tin, may be supplied with a hinging lid also of tin that swings over and closes the opening of the container. A solid lid, hinged or separate, is useful for closing off the fragrance delivery from the scented crystals when scenting is not desired (e.g. after retiring to bed, or when changing the mood in the room, etc.).

The optional lid may also be slotted or grated in such a way that when it is used to cover the opening of the container the opening is not completely sealed, thus allowing fragrance to permeate out from the container and into the room to be scented. A grill or grate structure on the lid may provide some child resistance by simply restricting complete physical access to the scented crystals. Thus the air freshener could still be used around small children. Grills or grates allow an infinite possibility for design. The only requirement is that the lid includes at least one opening such as a hole or slot. A plurality of holes or slots may be arranged in any pattern, such as to form a grate. Holes may be very tiny or up to the size where a small finger is just precluded from poking through to reach the crystals. A second material may be added to the lid, such as a piece of cloth that covers a larger hole(s) or slot(s). For example, the kit may include a canning jar having only a fabric cover retained by a metal screw retaining ring. Of course, the way in which a child-resistant grilled or grated lid attaches to the opening of the container is important. A threaded arrangement will likely ensure better child resistance than a snap-on lid, although if the friction-fit is tight enough, even a snap on structure suffices to deter handling of the crystals.

The Carton

Lastly, to complete the kit of the present invention, both the bag containing the plurality of fragranced salt crystals and the container are provided inside a carton, wherein the carton preferably includes a view window that aids in merchandizing. It is most preferred that the carton be a paperboard carton that is folded up from a blank. A gabled carton is preferred in that the high gabled portion of the carton may hold most of the bag of crystals. For example, the container may be placed in the assembled carton first, with its bottom against the inside bottom of the carton. Next, the bag of fragranced crystals may be placed on top of the container, perhaps with some of the bag inserted into the container and the remaining bulk of the bag staying up in the gable of the carton. In this way, the consumer will see the container through the window of the carton, and may also see (depending if both the jar and the bag of crystals are transparent) some of the colored salt crystals to get an idea of what the assembled salt crystal air freshener will look like. Some examples of gabled cartons include those described in U.S. Pat. No. 5,230,463 (Brauner) and U.S. Pat. No. 3,722,782 (Collie), the styles of which are useful for the kit of present invention. The viewing window may be cut into at least one of the panels and may be any particular shape (square, rectangular, oval, round, scalloped, or forming some other decorative outline). For example, a preferred window may comprise part of the front and part of one of the side panels of the carton. There may also be more than one window. The view window(s) may be completely open or may be covered from behind with clear or colored cellophane film. The preferred shape of the carton is approximately cuboid and should be about 2 inch square up to about 10 inches square. When a gable is featured as part of a cuboid carton, the carton is preferably about 4 to about 12 inches tall. A tab may be provided on one of the flaps of the gable panel and a corresponding slot such that the carton can be re-closed by inserting the tab in the slot provided on the gable panels.

The carton in the present invention may be useful as a unit for shipping and merchandizing. Alternatively, a number of individual kits may be placed within a larger, sturdier shipping carton that can be palletized.

Method of Assembling a Passive Air Freshener

In using the kit of the present invention, the consumer selects the product based on its aesthetic impression on the merchant's shelf or other want or need for a decorative and passive air freshener. A gabled carton gives height and flair to the kit, with the optional viewing window showing at least the decorative aspects (if any) of the container and perhaps part if not all of the package of crystals (to show the consumer the color of the dyed crystals and an idea of what the assembled air freshener will look like). Once having selected the kit, the consumer opens the carton, for example by pulling apart a glued flap or panel, or if gabled pulls apart a seam on the top of the gable of the box. Once inside the box, the consumer removes both the package containing the plurality of fragranced crystals and the container. The container is placed in the area to be fragranced (e.g. a room in the home, business, or institution), the package of crystals is opened (e.g. by ripping a tear strip, tearing along a precut slot in a heat-crimped area, pulling apart heat-sealed seams, or opening up a recloseable zipper, or the like), and then an amount of fragranced crystals are poured into the opening of the container to the level desired or as recommended in the instructions optionally provided. If a feature so provides, the packaging for the crystals may be resealed to store excess fragranced crystals for a future filling of the container, or the bag may be disposed of if empty. Lastly, and if provided, a decorative and/or safety grilled or grated lid is snapped, screwed on or otherwise attached over the opening of the container to secure the crystals therein, and/or to mitigate physical contact with the crystals. An optional lid without vapor slots may be provided such that the consumer has the option of simply closing off the jar so that the fragrance delivery to the room stops. When the fragrance is no longer detected from the salt crystals (e.g., after about 5 to 45 days), the consumer may dispose of the used crystals and refill the container with fresh crystals. As mentioned, a decorative container connotes refilling, and in that case the consumer would likely purchase refill pouches of crystals, using these to refill the container as needed. The refillable container may be washed out prior to refilling with new fragranced salt crystals.

Optional Lighting

The kit of the present invention may also include a small light for illuminating the fragranced salt crystals. Since the fragranced crystals described herein have optical beauty, a light shining through the crystals heightens that beauty, especially if the crystals are dyed with color. For example, a small self-contained LED light source with a battery may be supplied for placement in the container under the crystals. In a preferred embodiment, the consumer would first turn on the light source, place it in the bottom of the container, and then cover the light source with the crystals. Optionally the light source may be part of a coaster, base unit, or stand configured and dimensioned such that the container can fit upon it and be illuminated by the light from beneath. In this way the light source may shine up through the bottom of the container of crystals to illuminate both the container and the crystals therein. A light positioned exterior to the container allows the option of an electrical power cord (100 v, 220 v, etc.) rather than batteries. Larger base units or stands for the container may contain an incandescent, fluorescent, LED, or other appropriate lamp. The bulbs may be clear or colored as needed. There is also the option of changing color, for example with a rotating color wheel mounted in the base unit situated beneath the container. The lighting effect is most pronounced when the container is made from glass, or at least from transparent plastic.

We have thus described a unique and new kit for assembling a passive air freshener comprising a plurality of fragranced salt crystals provided in a bag, an open container and a carton for including both. The kit allows the consumer to assemble an air freshener comprising fragranced salt crystals within a container, with option for a grate or closable lid, and further option for a lighting source to illuminate the crystals.

We claim:

1. A kit for assembling a passive air freshener, said kit comprising:
a. a package containing a plurality of fragranced salt crystals, said crystals further comprising:
i. salt crystals grown from evaporation of a brine solution, said crystals sieved to distribute between about ⅜ inch and about 1 inch U.S. Standard sieves;
ii. fumed silica;
iii. a nonionic emulsifier; and,
iv. a fragrance oil.
b. a container having a bottom, sides and an open top, said container having a height to width ratio of from about 1:2 to about 3:1; and,
c. a carton,
wherein said carton encloses both said package and said container.

2. The kit of claim 1, wherein said salt crystals are sea or lake salt crystals, with said brine solution comprising ocean sea water or Great Salt Lake water.

3. The kit of claim 1, wherein said nonionic emulsifier is selected from the group consisting of sorbitan esters, alkoxylated sorbitan esters, C₂-C₆ glycols, glycol esters, glycerin, glyceryl esters, alkoxylated glyceryl esters, amide waxes, fatty alcohols, monoalcohol esters, polyethylene glycol, polypropylene glycol, polyethylene glycol esters, polypropylene glycol esters, fatty alcohol alkoxylates, alkyl phenol alkoxylates, alkoxylated fatty acid esters, alkanolamides, amine N-oxides, and alkylpolyglycosides, and mixtures thereof.

4. The kit of claim 1, wherein said fumed silica is untreated hydrophilic silicon dioxide of sub-micron size.

5. The kit of claim 3, wherein said nonionic emulsifier is an alkoxylated sorbitan ester.

6. The kit of claim 5, wherein said alkoxylated sorbitan ester is selected from the group consisting of ethoxylated sorbitan monooleate, ethoxylated sorbitan monolaurate, ethoxylated sorbitan monopalmitate, and ethoxylated sorbitan monostearate, and mixtures thereof.

7. The kit of claim 1, wherein said crystals further include a solvent selected from the group consisting of ethanol, methanol, isopropanol, n-propanol, methylpropanediol, ethylene glycol, propylene glycol, diethylene glycol monoalkyl ether, and dipropylene glycol monoalkyl ether, and mixtures thereof.

8. The kit of claim 1, wherein said crystals further include a preservative selective from the group consisting of antioxidants, uv absorbers, and antimicrobials, and mixtures thereof.

9. The kit of claim 1, wherein said crystals further include a colorant.

10. The kit of claim 1, wherein said carton further includes a view window.

11. The kit of claim 1 further including a lid appropriately dimensioned and configured to fit over the opening of said container.

12. The kit of claim 1 further including a light source.

13. The kit of claim 12, wherein said light source comprises a battery powered LED light dimensioned to fit inside and at the bottom of said container.

14. The kit of claim 12, wherein said light source comprises an 110 v or 220 v powered LED, fluorescent, or incandescent lamp mounted within a base unit, and wherein said container fits on top of said base unit and is illuminated from beneath by said light source.

15. The kit of claim 1 further including instructional literature.

16. A method of preparing the air freshener kit of claim 1, said method comprising the steps of:
a. mixing fumed silica, a nonionic emulsifier and fragrance oil to form a fragrance premix;
b. sourcing salt crystals grown from the evaporation of a brine solution;
c. sieving said salt crystals across 1 inch and ⅜ inch Standard U.S. Sieves;
d. tumbling said premix with said sieved salt crystals until said crystals are evenly coated with said premix;
e. sealing a plurality of said coated crystals within a package;
f. sourcing an open container dimensioned to hold a plurality of said crystals; and
g. placing said package of said crystals along with said container inside a carton.

17. A method of assembling a passive air freshener combining the steps of:
a. obtaining the kit of claim 1;
b. removing both said container and said package from said carton;
c. opening said package;
d. pouring a portion of fragranced crystals into said container; and
e. optionally resealing said package of crystals.

18. The method of claim 16, wherein said salt crystals are sea salt crystals obtained from the evaporation of Atlantic, Pacific or Caribbean waters.

19. The method of claim 16, wherein said nonionic emulsifier is selected from the group consisting of ethoxylated sorbitan monooleate, ethoxylated sorbitan monolaurate, ethoxylated sorbitan monopalmitate, and ethoxylated sorbitan monostearate, and mixtures thereof.

20. The method of claim 17, wherein said carton further includes a view window.
